(19) United States
(12) Patent Application Publication
(10) Pub. No.: US 2025/0332396 A1
(43) Pub. Date: Oct. 30, 2025

(54) DIRECT DRIVE ADJUSTABLE STROKE TATTOO MACHINE

(71) Applicant: CRITICAL TATTOO SUPPLY, LLC, Orange, CA (US)

(72) Inventor: Rodney Aguiar, Reno, NV (US)

(73) Assignee: CRITICAL TATTOO SUPPLY, LLC, Orange, CA (US)

(21) Appl. No.: 19/188,339

(22) Filed: Apr. 24, 2025

Related U.S. Application Data
(60) Provisional application No. 63/640,062, filed on Apr. 29, 2024.

(51) Int. Cl. A61M 37/00

(52) U.S. Cl. CPC A61M 37/0076; A61M 2205/106; A61M 2205/8206

(57) ABSTRACT

The device is an adjustable stroke tattoo machine, comprising: a drive motor, which comprises a drive shaft; a drive cam that is configured to matingly engage with the drive shaft; a connecting rod that is configured to engage with the drive cam; a stroke adjustment device, wherein the stroke adjustment device comprises a guide arm and a rack platform, which comprises a rack; and a plunger bar, which is configured to engage with one or more tattoo needles and which is configured to engage with the guide arm; wherein the guide arm is configured to engage with the connecting rod and which is configured to engage the rack platform; wherein the drive motor is configured to receive power from a power source; and the stroke adjustment device is configured to adjust a stroke length of the tattoo machine. The stroke adjustment device may adjust the stroke length electronically and/or, manually.

### CROSS-REFERENCE TO RELATED APPLICATIONS

This application claims the benefit of U.S. Provisional Patent Application No. 63/640,062, filed on Apr. 29, 2024, titled “DIRECT DRIVE ADJUSTABLE STROKE TATTOO MACHINE”, the contents of which are expressly incorporated herein by this reference, and to which priority is claimed.

### FIELD OF DISCLOSURE

The present disclosure relates to tattoo machines. More specifically, the present disclosure relates to a direct drive tattoo machine that has an adjustable stroke to make the stroke longer or shorter. Preferably, the tattoo machine may have at least three different stroke lengths that can easily be switched between by the user.

### BACKGROUND

Current tattoo machines do not offer precision and ease in adjusting the stroke length. U.S. Pat. No. 8,522,647 discloses a tattoo machine that has an adjustable offset cam. By adjusting where the connecting rod is on this offset cam, the stroke is adjusted. This is merely changing the “offset” of the offset cam and requires a set screw to lock the adjustment in place.

U.S. Pat. No. 11,260,209 discloses a microneedling machine for tattooing is disclosed, which has a substantially radially symmetric exterior shape and may include subcomponents that can be disassembled and sterilized. The machine may include an adjustment mechanism for configuring the impact of the microneedle into skin. However, the adjustment mechanism does not allow for a change in stroke length, only in stroke power.

Thus, what is needed is a tattoo machine that allows for an easy and precise adjustment of the stroke length, manually or automatically.

### SUMMARY OF THE INVENTION

To minimize the limitations in the cited references, and to minimize other limitations that will become apparent upon reading and understanding the present specification, the present disclosure relates to a tattoo or microneedling machine that has a fixed offset cam that rotates to turn rotational motion to linear motion and that adjusts the stroke length of that linear motion by using a pivot linkage. The linkage is adjusted in or out to change the leverage or ratio of the fixed offset cam to the linkage. The further out the linkage goes, the longer the stroke becomes, and the further it goes inward, the shorter the stroke becomes. The stroke length is adjustable on the fly with no setscrews. It may be adjusted manually through an adjustment ring/dial, or it may be adjusted through a small electric motor connected to the battery.

The stroke adjustment mechanics allow the stroke length to be adjusted while the tattoo machine is running, either in use or idling, in addition to being adjustable when the tattoo machine is off.

The tattoo machine of the present disclosure may include a gear box that may include the stroke adjustment mechanics. The stroke adjustment mechanics allow the user to change the stroke length of the plunger bar, which is connected to the removeable needle or cartridge. Once the stroke length is adjusted by the stroke adjustment mechanics, the plunger bar may be actuated in a linear back and forth motion by the connecting rod, which may be pushed back-and-forth by the drive cam, which may translate the rotational motion of the drive shaft into a back-and-forth motion of the connecting rod. The drive shaft may be connected to the drive motor, which is a rotational direct drive motor.

The gears that are part of the gear-drive that is used to adjust the stroke length may matingly connects to the stroke adjustment mechanics. A first gear may be matingly engaged with the gear shaft of the gear reduction motor, sometimes referred to gear drive motor. When the user wants to change the length of the stroke of the tattoo machine, the user actuates the gear reduction motor (or manually turns a dial), which may turn the gear shaft of the gear reduction motor, which turns a gear which, through the gear-drive, causes the first gear to turn and adjust the stroke adjustment mechanics. The tattoo machine may have a threaded and removeable grip.

The stroke adjustment mechanism may work like a rack and pinion steering, in that it may have a rack that is moved from side to side by a gear. The rack may be connected to plate, which is moved from side to side (left to right and vice versa). In the current embodiment, when the plate is moved to the left, the stroke of the plunger bar is longer and when the plate is moved to the right, the stroke is shorter. The system works in a rack and pinion manner. The connecting rod and the plunger may be connected via a plunger cam, which is attached to the rack plate, which moves back and forth (or up and down) in order to shorten or lengthen each stroke of the plunger bar. The plunger cam may be adjusted on the rack plate/gear box to be more forward (towards the needle tip) or backward (towards the motor and power connection).

In one embodiment, the battery may be integrated into the tattoo machine. This means that the controls for the motor voltage and the stroke adjustment may be part of a non-removable portion. Further, in another embodiment, the battery is removeable, but the controls are not removeable from the tattoo machine.

In alternate embodiments, the stroke adjustment device, components, and/or mechanism may be a micro linear actuator, which is a small actuator that creates linear motion, a ball screw, which is a threaded rod and threaded nut to create linear motion, and/or a pivot linkage, which is a linkage used to create linear motion.

One embodiment may be a tattoo machine, comprising: a drive motor, which comprises a drive shaft; a cam that is configured to matingly engage with said drive shaft; a connecting rod that is configured to engage with said drive cam; a stroke adjustment device, wherein said stroke adjustment device comprises a guide arm and a rack platform, which comprises a rack; and a plunger bar, which is configured to engage with one or more tattoo needles, and which is configured to engage with said guide arm; wherein said guide arm is configured to engage with said connecting rod and which is configured to engage said rack platform; wherein said drive motor is configured to receive power from a power source; wherein said stroke adjustment device is configured to adjust a stroke length of said tattoo machine. The stroke adjustment device may adjusts the stroke length electronically, manually, and or/a combination of both. The guide arm may be hingedly connected to the rack platform, such that when the connecting rod may be activated by the drive motor, the guide arm reciprocatingly moves the plunger bar in a linear manner. The stroke adjustment device may further comprise: a drive gear motor, which may be configured to receive power from the power source; and a rack gear drive; wherein the drive gear motor comprises a bottom gear; wherein the rack gear drive comprises a first gear and a second gear; wherein the bottom gear may be configured to matingly engage with the first gear; wherein the second gear may be configured to matingly engage with the rack; and wherein the drive motor gear may be configured adjust the stroke length, such that when the drive motor gear rotates the bottom gear rotates, which in turn rotates the first gear, which in turn rotates the second gear, which in turn drives the rack, which moves the rack platform, which in turn repositions the guide arm, such that the stroke length may be lengthened or shortened. The drive motor gear may be configured to accept one or more instructions from a user or a program to adjust the stroke length. The power source may be a battery; and the one or more instructions to adjust the stroke length may be provided by the battery, which may be operatively connected to the tattoo machine. The stroke adjustment device further comprises: a stroke adjustment ring; a gear drive; and a rack gear drive; wherein the stroke adjustment ring comprises a plurality of stroke adjustment ring gear teeth; wherein the gear drive comprises a top gear and a bottom gear; wherein the rack gear drive comprises a first gear and a second gear; wherein the stroke adjustment ring gear teeth may be configured to matingly engage with the top gear; wherein the bottom gear may be configured to matingly engage with the first gear; wherein the second gear may be configured to matingly engage with the rack; and wherein the stroke adjustment ring may be configured to be turned to adjust the stroke length, such that when the stroke adjustment ring may be rotated, and thus the stroke adjustment ring gear teeth may be rotated, the stroke adjustment ring gear teeth rotates the top gear, which in turn rotates the bottom gear, which in turn rotates the first gear, which in turn rotates the second gear, which in turn drives the rack, which moves the rack platform, which in turn repositions the guide arm, such that the stroke length may be lengthened or shortened. The stroke adjustment device may further comprise: a stroke adjustment ring; a gear drive/drive gear motor; and a rack gear drive; wherein the stroke adjustment ring comprises a plurality of stroke adjustment ring gear teeth; wherein the a gear drive/drive gear motor comprises a top gear and a bottom gear; wherein the rack gear drive comprises a first gear and a second gear; wherein the stroke adjustment ring gear teeth may be configured to matingly engage with the top gear; wherein the bottom gear may be configured to matingly engage with the first gear; wherein the second gear may be configured to matingly engage with the rack; and wherein the stroke adjustment ring may be configured to be turned to manually adjust the stroke length, such that the stroke adjustment ring, and thus the stroke adjustment ring gear teeth, rotates, which in turn rotates the top gear, which in turn rotates the bottom gear, which in turn rotates the first gear, which in turn rotates the second gear, which in turn drives the rack, which moves the rack platform, which in turn repositions the guide arm, such that the stroke length may be lengthened or shortened; and wherein the gear drive/drive motor gear may be configured to electronically adjust the stroke length, such that the gear drive/drive motor gear rotates the bottom gear, which in turn rotates the first gear, which in turn rotates the second gear, which in turn drives the rack, which moves the rack platform, which in turn repositions the guide arm, such that the stroke length may be lengthened or shortened. The gear drive/drive motor gear may be configured to accept one or more instructions from a user or a program to electronically adjust the stroke length. The power source may be a battery; and wherein the one or more instructions to adjust the stroke length may be provided by the battery, which may be operatively connected to the tattoo machine.

Another embodiment may be an electronic stroke adjustment tattoo machine, comprising: a drive motor, which may comprise a drive shaft; a cam that may be configured to matingly engage with the drive shaft; a connecting rod that may be configured to engage with the drive cam; a stroke adjustment device, wherein the stroke adjustment device comprises a guide arm, a drive gear motor; a rack gear drive; and a rack platform; wherein the guide arm may be configured to engage with the connecting rod and which may be configured to engage the rack platform; a plunger bar, which may be configured to engage with one or more tattoo needles, and which may be configured to engage with the guide arm; wherein the drive motor may be configured to receive power from a power source; wherein the guide arm may be hingedly connected to the rack platform, such that when the connecting rod may be activated by the drive motor, the guide arm reciprocatingly moves the plunger bar in a linear manner; wherein the stroke adjustment device may be configured to adjust a stroke length of the tattoo machine. The rack platform may comprises a rack; wherein the drive gear motor comprises a bottom gear; wherein the rack gear drive may comprise a first gear and a second gear; wherein the bottom gear may be configured to matingly engage with the first gear; and wherein the second gear may be configured to matingly engage with the rack. The drive motor gear may be configured to adjust the stroke length, such that the drive motor gear rotates the bottom gear, which in turn rotates the first gear, which in turn rotates the second gear, which in turn drives the rack, which moves the rack platform, which in turn repositions the guide arm, such that the stroke length may be lengthened or shortened. The drive motor gear may be configured to accept one or more instructions from a user or a program to adjust the stroke length. The electronic stroke adjustment tattoo machine of claim 16, wherein the power source may be a battery; and wherein the one or more instructions to adjust the stroke length may be provided by the battery, which may be operatively connected to the tattoo machine.

Another embodiment may be a manual stroke adjustment tattoo machine, comprising: a drive motor, which comprises a drive shaft; a cam that may be configured to matingly engage with the drive shaft; a connecting rod that may be configured to engage with the drive cam; a stroke adjustment device, wherein the stroke adjustment device comprises a stroke adjustment ring, a gear drive, a rack gear drive; a guide arm, and a rack platform; wherein the guide arm may be configured to engage with the connecting rod and which may be configured to engage the rack platform; a plunger bar, which may be configured to engage with one or more tattoo needles, and which may be configured to engage with the guide arm; wherein the drive motor may be configured to receive power from a power source; wherein the guide arm may be hingedly connected to the rack platform, such that when the connecting rod may be activated by the drive motor, the guide arm reciprocatingly moves the plunger bar in a linear manner; and wherein the stroke adjustment device may be configured to adjust a stroke length of the tattoo machine. The rack platform may comprise a rack; wherein the stroke adjustment ring comprises a plurality of stroke adjustment ring gear teeth; wherein the gear drive comprises a top gear and a bottom gear; wherein the rack gear drive comprises a first gear and a second gear; wherein the stroke adjustment ring gear teeth may be configured to matingly engage with the top gear; wherein the bottom gear may be configured to matingly engage with the first gear; and wherein the second gear may be configured to matingly engage with the rack. The stroke adjustment ring may be configured to be turned to adjust the stroke length, such that the stroke adjustment ring, and thus the stroke adjustment ring gear teeth, rotates, which in turn rotates the top gear, which in turn rotates the bottom gear, which in turn rotates the first gear, which in turn rotates the second gear, which in turn drives the rack, which moves the rack platform, which in turn repositions the guide arm, such that the stroke length may be lengthened or shortened; and wherein an upper end of the stroke length may be adjusted or limited.

The various features and embodiments may be combined in multiple combinations so that any feature in the disclosure may be present in any combination of disclosure elements.

Other features and advantages inherent in the disclosed system and method for delivery metric analysis and notification, besides those which are claimed and disclosed, will become apparent to those skilled in the art from the following detailed description and its accompanying drawings.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps, which are illustrated. When the same numeral appears in different drawings, it refers to the same or like components or steps.

FIG. 1 is an illustration of a front view of one embodiment of the direct drive electronic adjustable stroke tattoo machine of the present disclosure.

FIG. 2 is an illustration of a front cross-sectional view of one embodiment of the direct drive electronic adjustable stroke tattoo machine of the present disclosure.

FIG. 3 is an illustration of a side view of one embodiment of the direct drive electronic adjustable stroke tattoo machine of the present disclosure.

FIG. 4 is an illustration of a side cross-section view of one embodiment of the direct drive electronic adjustable stroke tattoo machine of the present disclosure.

FIG. 5 is an illustration of another side cross-section view of one embodiment of the direct drive electronic adjustable stroke tattoo machine of the present disclosure.

FIG. 6 is an illustration of a front view of one embodiment of the direct drive electronic adjustable stroke tattoo machine with a battery connected.

FIG. 7 is an illustration of a side view of one embodiment of the direct drive electronic adjustable stroke tattoo machine with a battery connected.

FIG. 8 is an illustration of another side view of one embodiment of the direct drive electronic adjustable stroke tattoo machine with a battery connected.

FIG. 9 is an illustration of a front view of one embodiment of the direct drive manual adjustable stroke tattoo machine of the present disclosure.

FIG. 10 is an illustration of a front cross-sectional view of one embodiment of the direct drive manual adjustable stroke tattoo machine of the present disclosure.

FIG. 11 is an illustration of a side view of one embodiment of the direct drive manual adjustable stroke tattoo machine of the present disclosure.

FIG. 12 is an illustration of a side cross-section view of one embodiment of the direct drive manual adjustable stroke tattoo machine of the present disclosure.

FIG. 13 is an illustration of another side cross-section view of one embodiment of the direct drive manual adjustable stroke tattoo machine of the present disclosure.

FIG. 14 is an illustration of a bottom perspective view of one embodiment of the direct drive manual adjustable stroke tattoo machine with a battery connected.

FIG. 15 is an illustration of a top perspective view of one embodiment of the direct drive manual adjustable stroke tattoo machine with a battery connected.

FIG. 16 is an illustration of a bottom and front perspective view of one embodiment of an interior of the direct drive manual adjustable stroke tattoo machine.

FIG. 17 is an illustration of a front and side perspective view of one embodiment of an interior of the direct drive manual adjustable stroke tattoo machine.

FIG. 18 is an illustration of a front and other side perspective view of one embodiment of an interior of a direct drive adjustable stroke tattoo machine that may be adjusted manually or electrically.

FIG. 19 is an illustration of a front perspective view of one embodiment of an interior of a direct drive adjustable stroke tattoo machine that may be adjusted manually or electrically.

FIG. 20 is an illustration of a side perspective view of one embodiment of an interior of a direct drive adjustable stroke tattoo machine that may be adjusted manually or electrically.

FIG. 21 is an illustration of a front and side perspective view of one embodiment of an interior of a direct drive adjustable stroke tattoo machine that may be adjusted manually or electrically.

FIG. 22 is an illustration of a front view of another embodiment of a direct drive electronic adjustable stroke tattoo machine.

FIG. 23 is an illustration of a cross-section view of another embodiment of a direct drive electronic adjustable stroke tattoo machine.

FIG. 24 is an illustration of a latitudinal cross-section view of another embodiment of a direct drive electronic adjustable stroke tattoo machine.

FIG. 25 is an illustration of a perspective view of another embodiment of a direct drive adjustable stroke tattoo machine.

### DETAILED DESCRIPTION

In the following detailed description of various embodiments, numerous specific details are set forth in order to provide a thorough understanding of various aspects of the embodiments. However, these embodiments may be practiced without some or all of these specific details. In other instances, well-known methods, procedures, and/or components have not been described in detail so as not to unnecessarily obscure aspects of the embodiments.

While multiple embodiments are disclosed, still others will become apparent to those skilled in the art from the following detailed description. As will be realized, these embodiments are capable of modifications in various obvious aspects, all without departing from the spirit and scope of protection. Accordingly, the graphs, figures, and the detailed descriptions thereof, are to be regarded as illustrative in nature and not restrictive. Also, the reference or non-reference to a particular embodiment shall not be interpreted to limit the scope of protection.

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and/or “about” generally refer to a deviance of within 5% of the indicated number or range of numbers. In one embodiment, the term “approximately” and “about,” may refer to a deviance of between 0.0001-10% from the indicated number or range of numbers.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment.

“Optional” or “optionally” or “alternatively” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers, or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods, devices, and systems may be understood more readily by reference to the following detailed description of preferred embodiments and the examples included therein and to the Figures and their previous and following description.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments. It is to be understood that the methods and systems are not limited to specific methods, specific components, or to particular implementations. It is also to be understood that the terminology used herein is for the purpose of describing particular embodiments only and is not intended to be limiting.

The rack gear, gear drive motors, drive reduction motors, motor gears, gears, drive gears, gear drives, stroke adjustment devices, stroke adjustment dials, stroke adjustment rings, stroke adjustment device gears, and/or the other components that allow a user to, on the fly, adjust a stroke length of the tattoo machines of the present disclosure may sometimes be referred to collectively as the stroke adjustment device or the stroke adjustment components.

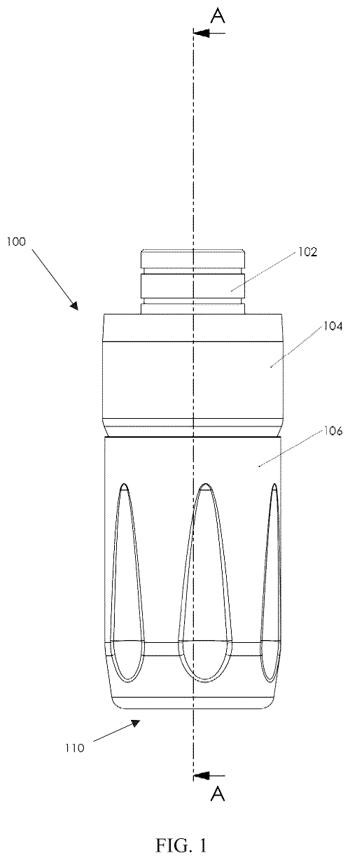

FIG. 1 is an illustration of a front view of one embodiment of the direct drive electronic adjustable stroke tattoo machine of the present disclosure. As shown in FIG. 1, tattoo machine 100 may comprise housing 104, removable grip 106, power connection end 102 and needle end 110. As shown, tattoo machine 100 may be a compact pen style tattoo or microneedling machine.

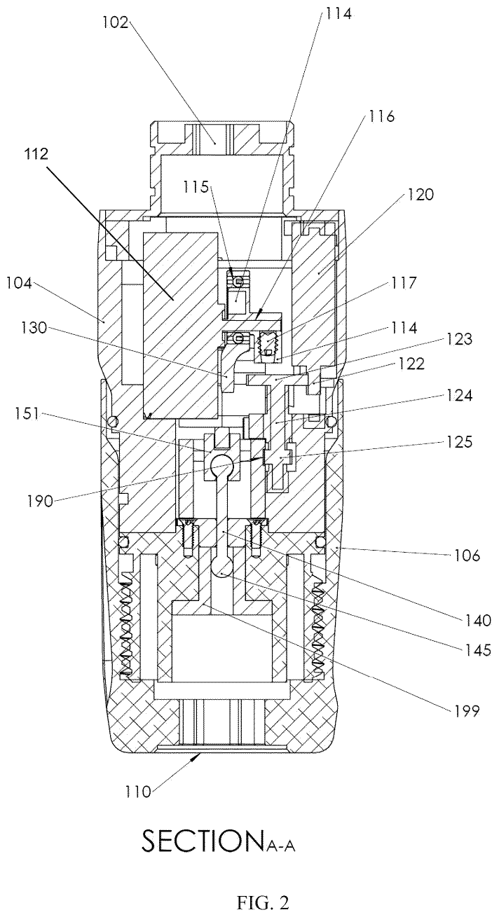

FIG. 2 is an illustration of a front cross-sectional view of one embodiment of the direct drive electronic adjustable stroke tattoo machine of the present disclosure. As shown in FIG. 2, tattoo machine 100 may comprise housing 104, connectors 105, removable grip 106, power connection end 102 needle end 110, drive motor 112 drive shaft 116, cam 114, cam bearing 115, drive shaft cam connector 117, connecting rod 130, gear drive motor 120, which may comprise motor gear 122 gear drive 124, which may comprise drive gear 123 and rack gear 125, rack platform 150, which may comprise rack 190 and guide arm 151, plunger bar 140, plunger bar end 145, and plunger channel 199.

As shown, drive motor 112 rotates drive shaft 116, which is connected to offset cam 114 via drive shaft cam connector 117, which causes offset cam 114 to rotate in an offset manner. Cam bearing 115, which is between cam 114 and connecting rod 130, allows tattoo machine 100 to begin the translation the rotational motion of drive motor 112 into linear motion via connecting rod 130 and the stroke adjustment components. Connecting rod 130 may be connected to guide arm 151, which may be operatively connected to plunger bar 140, such that when connecting rod 130 moves back and forth, guide arm 151 hinges back and forth, thus causing plunger bar 140 to move back and forth within plunger channel 199.

As shown, the gear drive motor 120 allows a user to automatically alter the length of the stroke of tattoo machine 100. When the user activates gear drive motor 120, motor gear 122 rotates. Motor gear 122 as shown, is interconnected with drive gear 123, which is rotated, such that gear drive 124 and rack gear 125 are rotated. Rack gear 125, as shown, is interconnected with rack 190, which is part of rack platform 150, such that when rack gear 125 is rotated, rack 190 and rack platform 150 move up and down in a linear manner. Guide arm 151 is connected to rack platform 150, such that as rack platform 150 moves up and down, the stroke of plunger bar 140 is lengthened or shortened.

FIG. 2 shows that removeable grip 106 may be threadedly connected to housing 104. In other embodiments, removable grip 106 may be connected to housing 104 via other devices, such as friction, snap, magnets, and the like.

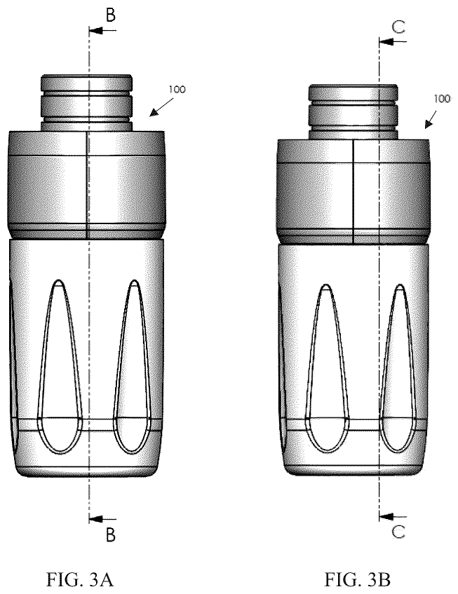

FIG. 3 is an illustration of a side view of one embodiment of the direct drive electronic adjustable stroke tattoo machine 100 of the present disclosure.

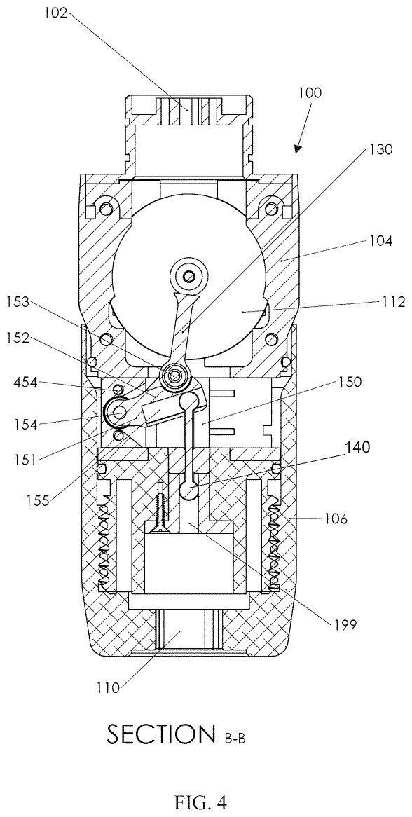

FIG. 4 is an illustration of a side cross-section view of one embodiment of the direct drive electronic adjustable stroke tattoo machine of the present disclosure. As shown in FIG. 4, tattoo machine 100 may comprise housing 104, removable grip 106, power connection end 102 needle end 110, drive motor 112 connecting rod 130, connecting rod connector 153, rack platform 150, which may comprise guide arm 151, guide arm hinge connector 154, and guide arm channel 155, and plunger bar 140.

As shown, connecting rod 130 may be connected to guide arm 151 via connecting rod connector 153, which may be a pin and ball bearing ring as shown. In other embodiments, rod connector 153 may be any low friction connector that allows guide arm 151 to be articulated by connecting rod 130 as connecting rod 130 moves back and forth. FIG. 4 shows that guide arm 151 is connected to rack platform 150 via guide arm hinge connector 154, which may be a pin and ball bearing ring as shown. In other embodiments, guide arm hinge connector 154 may be any low friction connector that allows guide arm 151 hinge with respect to rack platform 150 when articulated by connecting rod 130 as connecting rod 130 moves back and forth. As shown, guide arm 151 may be hingedly connected to rack platform 150, such that when connecting rod 130 is activated by drive motor 112 guide arm 151 reciprocatingly moves plunger bar 140 in a linear manner, thus completing the translation of rotation movement to linear movement.

FIG. 4 also shows that guide arm hinge connector 154 may have multiple connection points with rack platform 150, such as connection aperture 454. This allows tattoo machine 100 to be further modified to meet the needs of users and to conform with different types of needles and/or cartridges.

FIG. 4 shows that plunger bar 140 may be operatively connected to guide arm 151 at guide arm channel 155. Guide arm channel 155 may be configured such that when rack platform 150 is positioned to the left, as shown, plunger bar 140 has a stroke that is greater in length than when rack platform 150 is positioned to the right. The adjustment, via rack 190, may work like rack and pinion steering. As rack platform 150 is driven back and forth, the stroke length of plunger bar 140 gets longer or shorter, as desired by the user. As shown, plunger bar 140 may only move in an up and down manner, as guided and constrained by plunger channel 199.

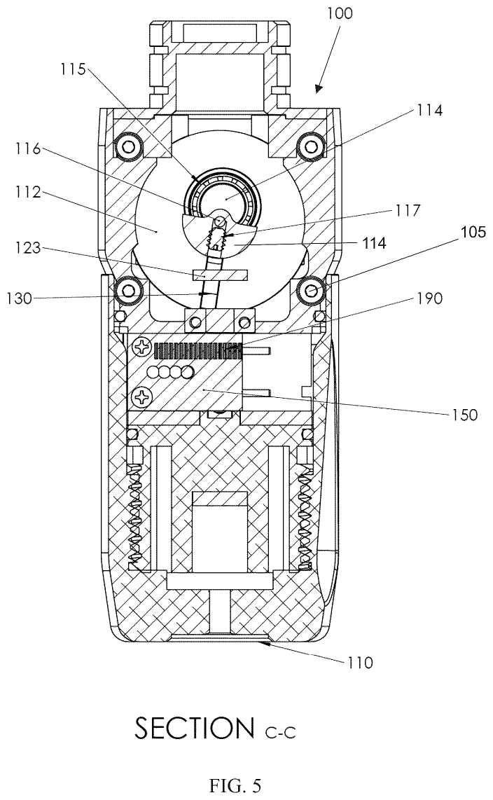

FIG. 5 is an illustration of another side cross-section view of one embodiment of the direct drive electronic adjustable stroke tattoo machine of the present disclosure. As shown in FIG. 5, tattoo machine 100 may comprise drive motor 112 drive shaft 116, cam 114 cam bearing 115, drive shaft cam connector 117, connecting rod 130, drive gear 123, connecting rod connector 153, and rack platform 150, which may comprise rack 190 and guide arm 151.

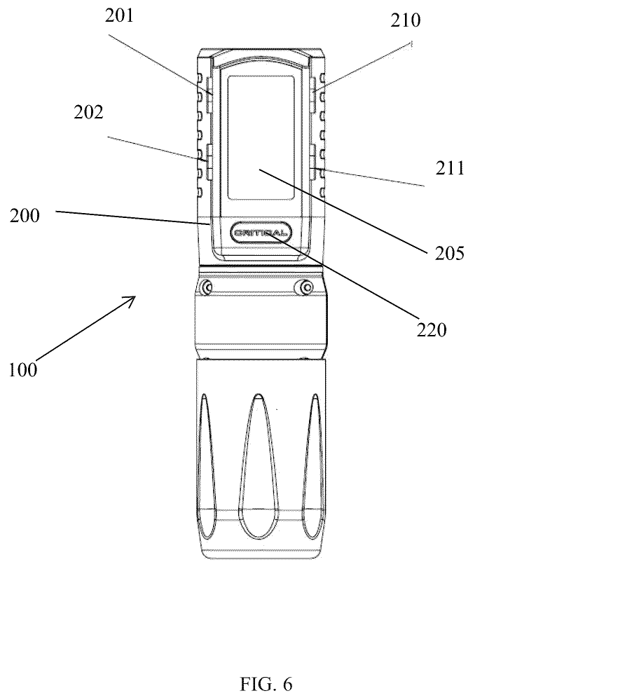

FIG. 6 is an illustration of a front view of one embodiment of the direct drive electronic adjustable stroke tattoo machine with a battery connected. As shown in FIG. 6, battery 200, or other power source and/or controller, may be operatively connected to tattoo machine 100. Battery 200 may comprise buttons 201, 202, 210, 211, 220, which may, as provided or as programmed by a user, allow a user to control the various functions of tattoo machine 100. In one embodiment, button 201 may be used to adjust the stroke up (shorten), button 202 may be used to adjust the stroke down (lengthen), button 210 may be used to increase the voltage, button 211 may be used to decrease the voltage, and button 220 may be used to power on and off tattoo machine or select various options via display 205. Depending on the desires and handedness of the user, buttons 201, 202, 210, 211 may be swapped and/or reprogrammed. The controller and/or power source may be a battery or a direct connection to a power supply or wall plug. The controller my be integrated with the battery, a wireless signal coming from a computer, such as a laptop or smart phone, or may be integrated with a wired power supply.

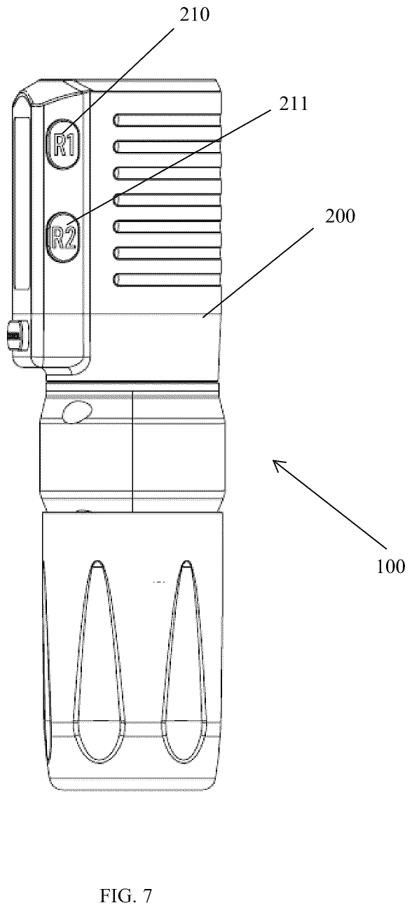

FIG. 7 is an illustration of a side view of one embodiment of the direct drive electronic adjustable stroke tattoo machine with a battery connected. As shown in FIG. 7, battery 200 is connected to tattoo machine 100 and may have two physical buttons 210, 211 on the right side that are labeled R1 and R2.

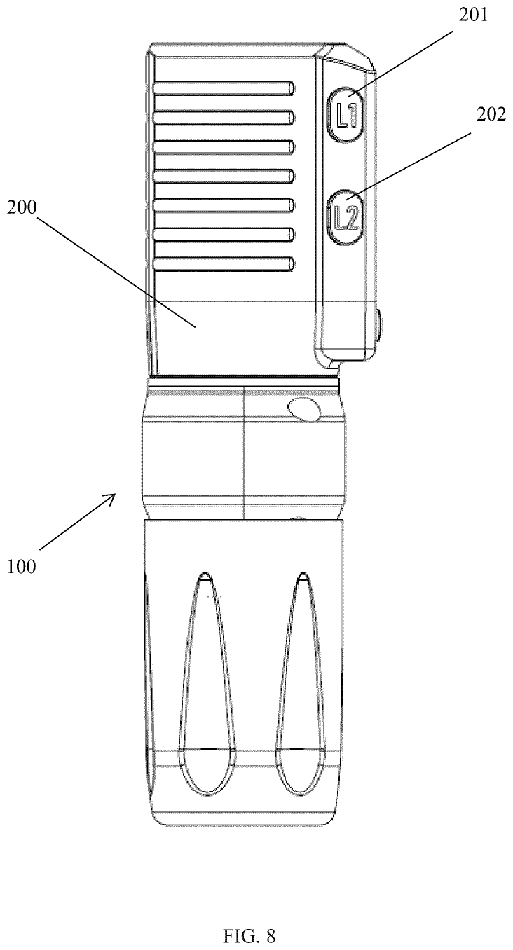

FIG. 8 is an illustration of another side view of one embodiment of the direct drive electronic adjustable stroke tattoo machine with a battery connected. As shown in FIG. 8, battery 200 is connected to tattoo machine 100 and has two physical buttons 201, 202 on the left side that are labeled L1 and L2.

In other embodiments, the battery or controller may have no physical buttons and instead may have soft keys or a remote control.

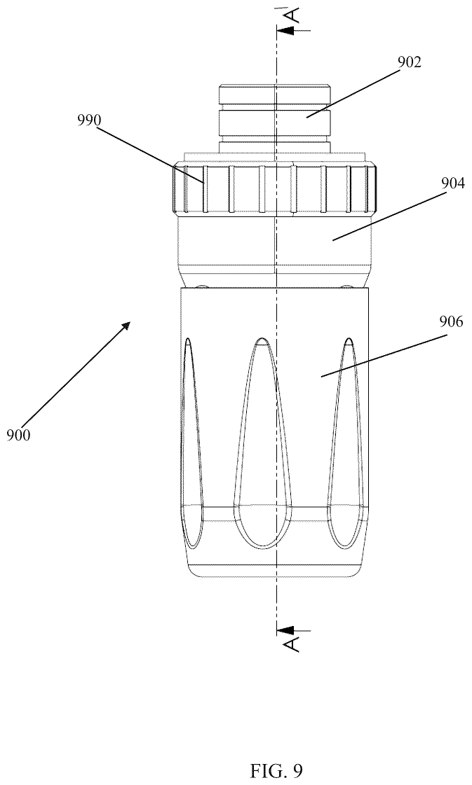

FIG. 9 is an illustration of a front view of one embodiment of the direct drive manual adjustable stroke tattoo machine of the present disclosure. As shown in FIG. 9, tattoo machine 100 may comprise stroke adjustment ring 990, housing 904, removable grip 906, power connection end 902 and needle end 910. As shown, stroke adjustment ring 990 may be a manual dial or ring that is configured to be easily gripped and turned/rotated/twisted by a user. In other embodiments, stroke adjustment ring 990 may be ring, dial, finger dial, scroller, button, knob, switch, manually turntable device, manual adjustment mechanism, tactile movement device, and the like. Stroke adjustment ring 990 may preferably be the outer and visible portion of the stroke adjustment device of the tattoo machine of the present disclosure, which is connected to one or more internal stroke adjustment components as shown and described herein.

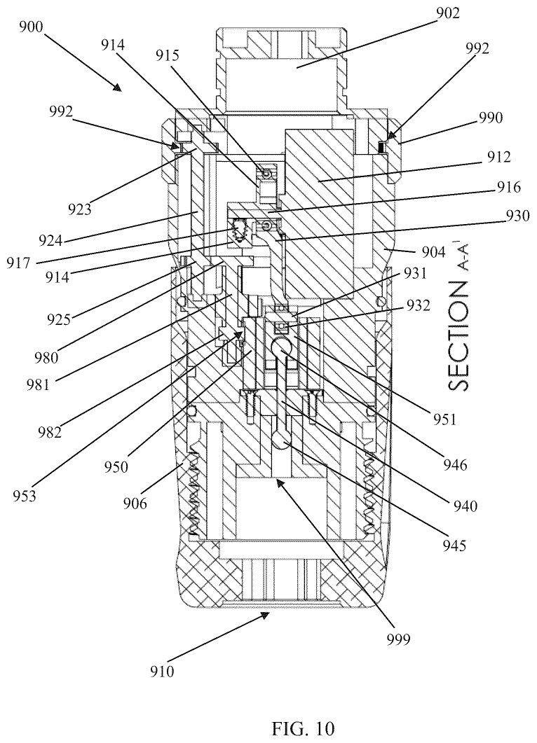

FIG. 10 is an illustration of a front cross-sectional view of one embodiment of the direct drive manual adjustable stroke tattoo machine of the present disclosure. As shown in FIG. 10, tattoo machine 900 may comprise housing 904, removable grip 106, power connection end 902 and needle end 910, drive motor 912 drive shaft 916, cam 914, cam bearing 915, drive shaft cam connector 917, connecting rod 930, stroke adjustment ring 990, which may comprise stroke adjustment ring gear teeth 992 gear drive 924, which may comprise bottom gear 923 and top gear 925, rack gear drive 981, which may comprise first gear 980 and second gear 982 rack platform 950, which may comprise rack 953 and guide arm 951, plunger bar 940, which may comprise plunger bar guide arm end 946 and plunger bar needle end 945, connecting rod pin connector 931 and bearing 932. In one embodiment the stroke adjustment ring gear teeth may be non-protruding interior surface, which is configured to matingly engage with a top gear, which may also not have teeth and may have a non-protruding exterior surface. In this manner the stroke adjustment device components may equivalently and matingly engage via friction rather than gear teeth.

As shown, drive motor 912 rotates drive shaft 916, which is connected to offset cam 914 via drive shaft cam connector 917, which causes cam 914 to rotate in an offset manner. Cam bearing 915, which is between cam 914 and connecting rod 930, allows tattoo machine 900 to translate the rotational motion of drive motor 912 into a mostly linear motion of connecting rod 930. Connecting rod 930 may be connected to guide arm 951 via connecting rod pin connector 931 and bearing 932. Guide arm 951, as shown, may be operatively connected to plunger bar 140 at plunger bar guide arm end 946, such that when connecting rod 930 moves back and forth, guide arm 951 hinges back and forth, thus causing plunger bar 940 to move back and forth within plunger channel 999.

As shown, stroke adjustment ring 990, and thus stroke adjustment ring gear teeth 992 may be manually turned or rotated by a user, to the left or to the right, which allows a user to manually alter the length of the stroke of tattoo machine 900. When the user manually turns stroke adjustment ring 990, then the stroke adjustment ring gear teeth 992 engage with and turn bottom gear 923 of gear drive 924. This causes top gear 925 to turn, which engages and turns first gear 980 of rack gear drive 981. This causes second gear 982 of rack gear drive 981 to turn, which engages with rack 953 of rack platform 950. This causes rack platform 950 to move, as shown, in an in and out manner. As rack platform 950 moves in and out, this changes the relative positioning of plunger bar 940 and guide arm 951, which causes the stroke of plunger bar 940 to lengthen or shorten.

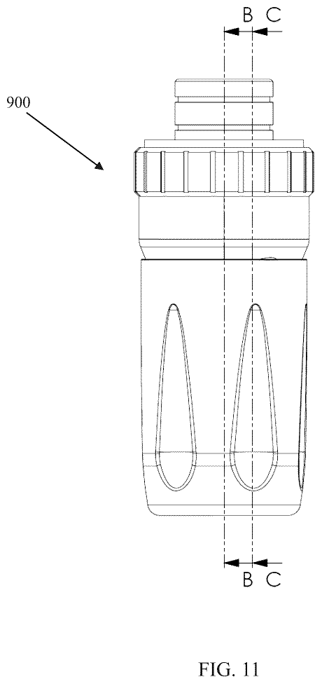

FIG. 11 is an illustration of a side view of one embodiment of the direct drive manual adjustable stroke tattoo machine 900 of the present disclosure.

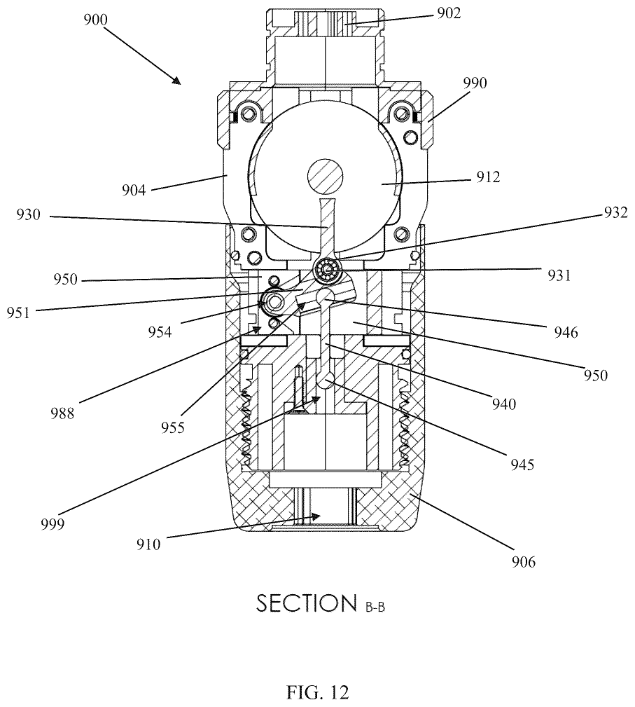

FIG. 12 is an illustration of a side cross-section view of one embodiment of the direct drive manual adjustable stroke tattoo machine of the present disclosure. As shown in FIG. 12, tattoo machine 900 may comprise housing 904, removable grip 906, power connection end 902 needle end 910, drive motor 912 connecting rod 930, connecting rod pin connector 931, bearing 932 rack platform 950, which may comprise guide arm 951, guide arm hinge connector 954, and guide arm channel 955, plunger bar 940, which may comprise plunger bar guide arm end 946 and plunger bar needle end 945, plunger channel 999, and stroke adjustment ring 990.

As shown, connecting rod 930 may be connected to guide arm 951 via connecting rod pin connector 931 and bearing 932 which may be a pin and ball bearing ring as shown. In other embodiments the connecting rod connector may be any low friction connector that allows guide arm 951 to be articulated by connecting rod 930 as connecting rod 930 moves up and down. FIG. 12 shows that guide arm 951 may be connected to rack platform 950 via guide arm hinge connector 954, which may be a pin and ball bearing ring as shown. In other embodiments, guide arm hinge connector 954 may be any low friction connector that allows guide arm 951 hinge with respect to rack platform 950 when articulated by connecting rod 930 as connecting rod 930 moves up and down. As shown, guide arm 951 may be hingedly connected to rack platform 950, such that when connecting rod 930 is activated by drive motor 912 guide arm 951 reciprocatingly moves plunger bar 940 in a linear manner.

FIG. 12 also shows that guide arm hinge connector 954 may have multiple connection points, in this case holes, on rack platform 950. This allows tattoo machine 900 to be further modified to meet the needs of users and conform with different types of needles and cartridges.

FIG. 12 shows that plunger bar 940 may be operatively connected to guide arm 951 at guide arm channel 955. Guide arm channel 955 may be configured such that when rack platform 950 is positioned to the left, as shown, plunger bar 940 has a stroke that is greater in length than when rack platform 950 is positioned to the right. The adjustment, via rack 953, may work like rack and pinion steering. As rack platform 950 is driven back and forth, the stroke length of plunger bar 940 gets longer or shorter, as desired by the user. As shown, plunger bar 940 may preferably only move in an up and down manner, as guided and constrained by plunger channel 999.

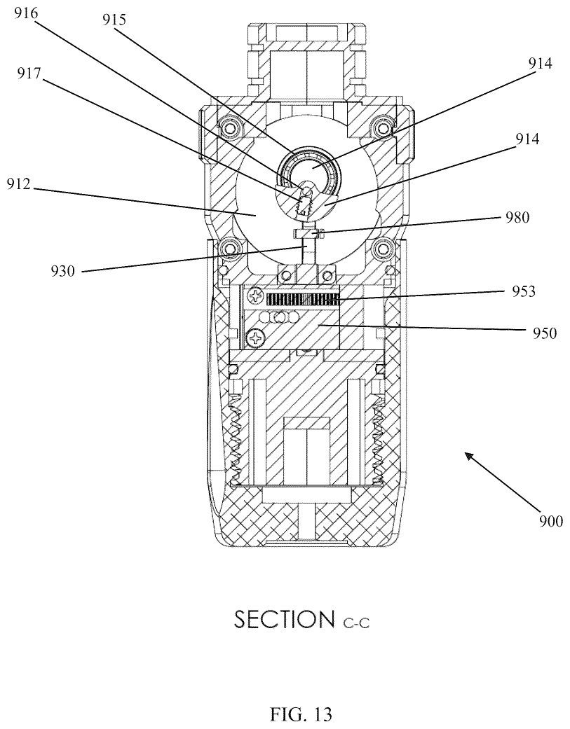

FIG. 13 is an illustration of another side cross-section view of one embodiment of the direct drive manual adjustable stroke tattoo machine of the present disclosure. As shown in FIG. 13, tattoo machine 900 may comprise drive motor 912 drive shaft 916, cam 914 cam bearing 915, drive shaft cam connector 917, connecting rod 930, first gear 980, and rack platform 950, which may comprise rack 953 and guide arm 151.

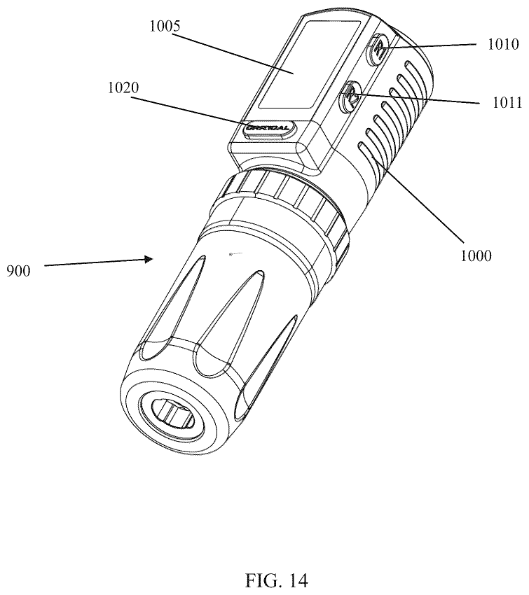

FIG. 14 is an illustration of a bottom perspective view of one embodiment of the direct drive manual adjustable stroke tattoo machine with a battery connected. As shown in FIG. 14, battery 1000, or other power source and controller, may be operatively connected to tattoo machine 900. Battery 1000 may comprise buttons 1010, 1011, 1020, which may, as provided or as programmed by a user, allow a user to control the various functions of tattoo machine 900. In one embodiment, button 1010 may be used to increase the voltage, button 1011 may be used to decrease the voltage, and button 1020 may be used to power on and off tattoo machine or select various options via display 1005. Depending on the desires and handedness of the user, buttons 1010, 1011 may be swapped and/or reprogrammed.

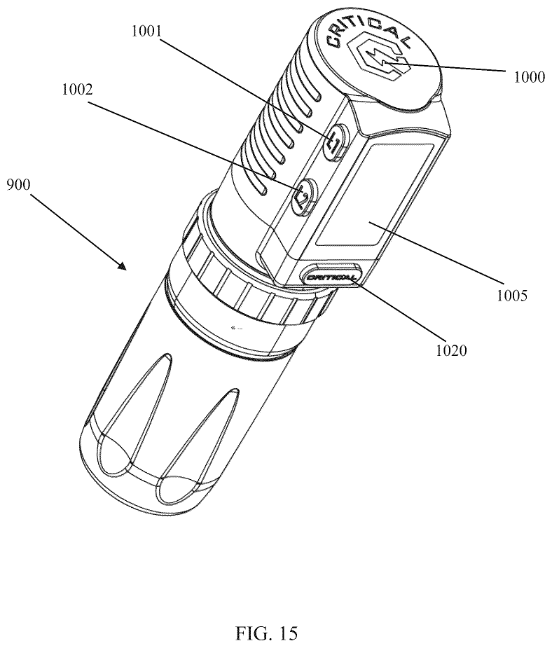

FIG. 15 is an illustration of a top perspective view of one embodiment of the direct drive manual adjustable stroke tattoo machine with a battery connected. As shown in FIG. 15, battery 1000, or other power source and controller, may be operatively connected to tattoo machine 900. Battery 1000 may comprise buttons 1001, 1002, 1020, which may, as provided or as programmed by a user, allow a user to control the various functions of tattoo machine 900. In one embodiment, button 1010 may be used to increase the voltage, button 1011 may be used to decrease the voltage, and button 1020 may be used to power on and off tattoo machine or select various options via display 1005. Depending on the desires and handedness of the user, buttons 1001, 1002 may be swapped and/or reprogrammed. Although tattoo machine 900 adjusts the length of the stroke manually, in some embodiments the tattoo machine of the present disclosure may work both automatically and manually. Accordingly, one or more buttons 1001, 1002, 1010, 1011, may be programmed to automatically adjust a stroke to be longer or shorter.

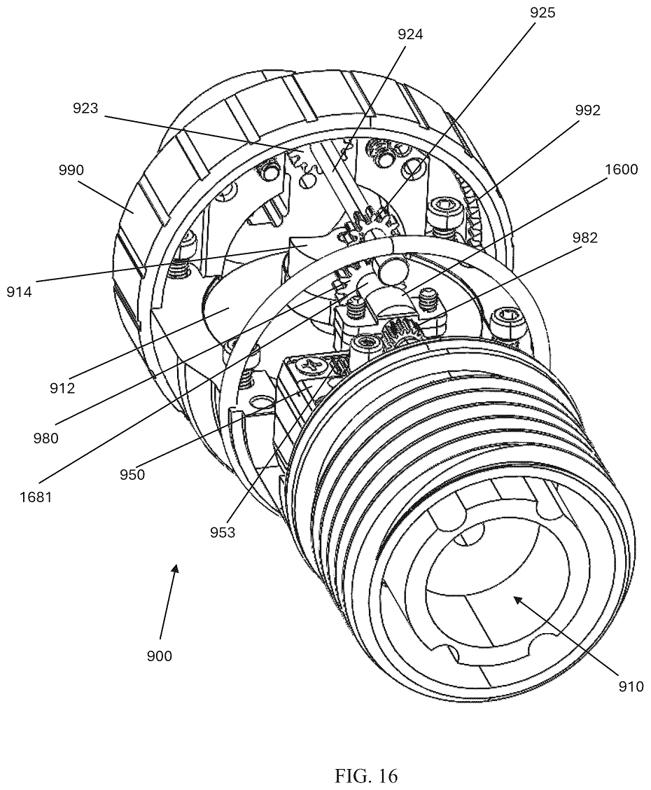

FIG. 16 is an illustration of a bottom and front perspective view of one embodiment of an interior of the direct drive manual adjustable stroke tattoo machine. As shown in FIG. 16, tattoo machine 900 is shown with the grip removed and most of the housing cut away. As shown, tattoo machine 900 may comprise needle end 910, drive motor 912 cam 914, stroke adjustment ring 990, which may comprise stroke adjustment ring gear teeth 992 gear drive 924, which may comprise bottom gear 923 and top gear 925, rack gear drive sheath 1681, first gear 980, second gear 982 and rack platform 950, which may comprise rack 953. FIG. 16 shows how a turn of stroke adjustment ring 990 causes rack platform 950 to move back and forth, which in turn adjusts the stroke length of tattoo machine 900. Although two gear drives 924, 981 are shown, there may be one gear drive, or there may be three or more gear drives, used in order to translate a motion or action into an adjustment of the stroke length.

As shown, stroke adjustment ring 990, and thus stroke adjustment ring gear teeth 992 may be manually turned or rotated by a user, to the left or to the right, which allows a user to manually alter the length of the stroke of tattoo machine 900. When the user manually turns stroke adjustment ring 990, then the stroke adjustment ring gear teeth 992 engage with and turn bottom gear 923 of gear drive 924. This causes top gear 925 to turn, which engages and turns first gear 980 of rack gear drive 981, which is covered by sheath 1681. This causes second gear 982 of rack gear drive 981 to turn, which engages with rack 953 of rack platform 950. This causes rack platform 950 to move, as shown, in back and forth (or right to left/left to right) manner. As rack platform 950 moves back and forth, this changes the relative positioning of plunger bar 940 and guide arm 951 (which are not shown in this view), which causes the stroke of plunger bar 940 to lengthen or shorten. FIG. 16 also shows how rack gear drive 981 may be supported and stabilized by housing 1600.

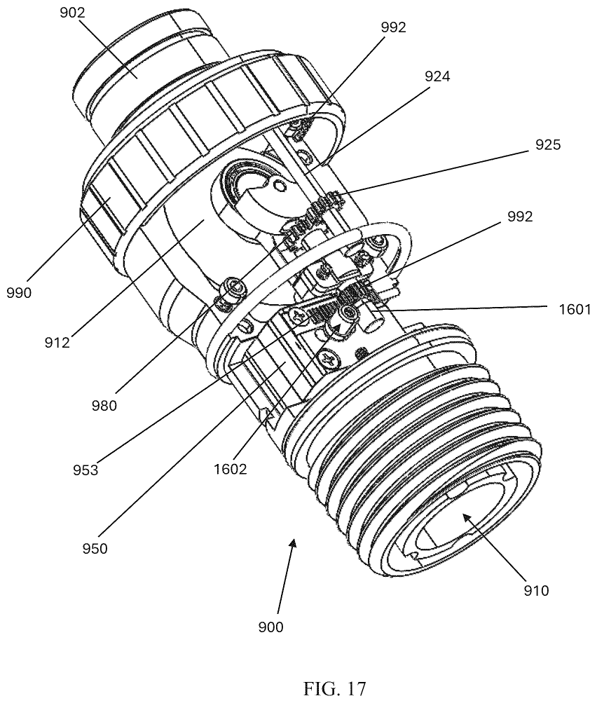

FIG. 17 is an illustration of a front and side perspective view of one embodiment of an interior of the direct drive manual adjustable stroke tattoo machine showing a rack limitation and adjustment system. As shown in FIG. 17, tattoo machine 900 is shown with the grip removed and most of the housing cut away. As shown, tattoo machine 900 may comprise power connection end 902, needle end 910, drive motor 912 stroke adjustment ring 990, which may comprise stroke adjustment ring gear teeth 992 gear drive 924, and which may comprise top gear 925, first gear 980, second gear 982 rack limiting nub 1601, and rack platform 950, which may comprise rack 953 and adjustable limitation device 1602. FIG. 17 shows how the stroke length adjustment may be limited to less than the full range of change in stroke length. In one embodiment, the stroke length may range from 1 mm to over 6 mm, which drives the removeable needles shallower (1 mm) or deeper (6 mm). Preferably, the stroke length may be adjusted from 2 to 4 mm. The embodiment shown in FIG. 17 may further limit the higher/longer range of the stroke, such that the stroke lengths may be from 2 mm to 2.5 mm, 3.0 mm, 3.5 mm, or some length greater than 2 mm, but less than 4 mm. The stroke length high end may be limited/adjusted by untightening and moving adjustable limitation device 1602 and then retightening it to rack platform 950 in a position that prevents the stroke length from reaching its mechanical maximum. As shown in FIG. 17, rack limiting nub 1601 may be at an end of rack gear drive 981 and may be configured to physically contact, and be impeded by, adjustable limitation device 1602 such that the high side of the stroke length may be limited. In this manner, a manufacturer or a user may adjust how long the longest stroke length may be. This capping of the stroke length is in addition to a user being able to, on the fly, adjust the stroke length of tattoo machine 900 to be any length between the high and low end of the stroke length range.

As shown, the turn of stroke adjustment ring 990 to the left causes rack platform 950 to move to the left, which lengthens the stroke of tattoo machine 900. When stroke adjustment ring 990 is turned to the right, this causes rack platform 950 to move to the right, which shortens the stroke of tattoo machine 900.

Although two gear drives 924, 981 are shown, there may be one or three or more gear drives used in order to translate a motion or action into an adjustment of the stroke length.

As shown, stroke adjustment ring 990, and thus stroke adjustment ring gear teeth 992 may be manually turned or rotated by a user, to the left or to the right, which allows a user to manually alter the length of the stroke of tattoo machine 900. When the user manually turns stroke adjustment ring 990, then the stroke adjustment ring gear teeth 992 engage with and turn bottom gear 923 of gear drive 924. This causes top gear 925 to turn, which engages and turns first gear 980 of rack gear drive 981. This causes second gear 982 of rack gear drive 981 (which is covered by sheath 1681) to turn, which engages with rack 953 of rack platform 950. This causes rack platform 950 to move, as shown, in back and forth (or right to left/left to right) manner. As rack platform 950 moves back and forth, this changes the relative positioning of plunger bar 940 and guide arm 951 (which are not shown in this view), which causes the stroke of plunger bar 940 to lengthen or shorten.

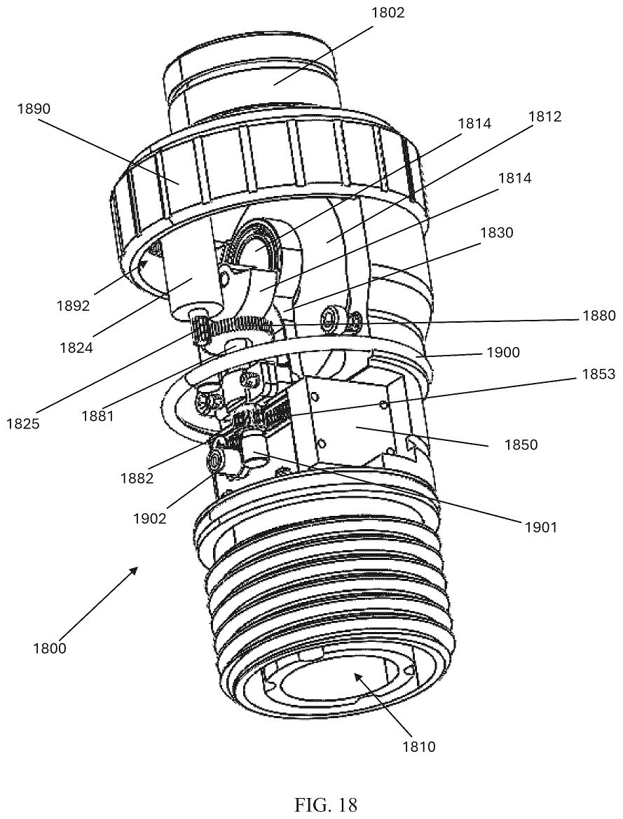

FIG. 18 is an illustration of a front and other side perspective view of one embodiment of an interior of a direct drive adjustable stroke tattoo machine that may be adjusted manually or electrically. As shown in FIG. 17, tattoo machine 1800 is shown with the grip removed and most of the housing cut away. As shown, tattoo machine 1800 may comprise power connection end 1802 needle end 1810, drive motor 1812 manual stroke adjustment ring 1890, which may comprise stroke adjustment ring gear teeth 1892, gear drive/drive gear motor 1824, and which may comprise a manual top gear (not shown in this view) and bottom gear 1825, rack gear drive, which is covered by rack gear drive sheath 1881 first gear 1880, second gear 1882 and rack limiting nub 1901, and rack platform 1850, which may comprise rack 1853 and adjustable limitation device 1902. FIG. 18 shows how the stroke length adjustment may be done automatically and/or manually. The user may either turn manual stroke adjustment ring 1890, thus causing rack platform 1850 to move back and forth, or the user may use a controller or battery button to automatically engage gear drive/drive gear motor 1824, thus causing rack platform 1850 to move back and forth. When rack platform 1850 moves back and forth, the stroke length of tattoo machine 1800 is moved from shorter to longer. Once the user sets the stroke length, automatically or manually, the stroke length is locked in, and the user may drive the needle or cartridge (at varying speeds depending on the voltage) during the process of tattooing. The user may pause, and instantly and on the fly, reset the stroke length, manually or automatically, lock in the new stroke length, and continue the process. Rack limiting nub 1601 and adjustable limitation device 1602 may allow a user or manufacturer to adjust the range of the stroke length to be less than the full range.

In other embodiments, adjustable limitation device 1602 may be a spring loaded pressure device (such as a spring-loaded ball detent) that holds rack platform 1850 in place once the user, via a controller and via gear motor 1824, adjusts the stroke length. In this embodiment, rack limiting nub 1601 pushes adjustable limitation device 1602 from one stroke length position to another, and then adjustable limitation device 1602 holds rack platform 1850 at the desired position/stroke length.

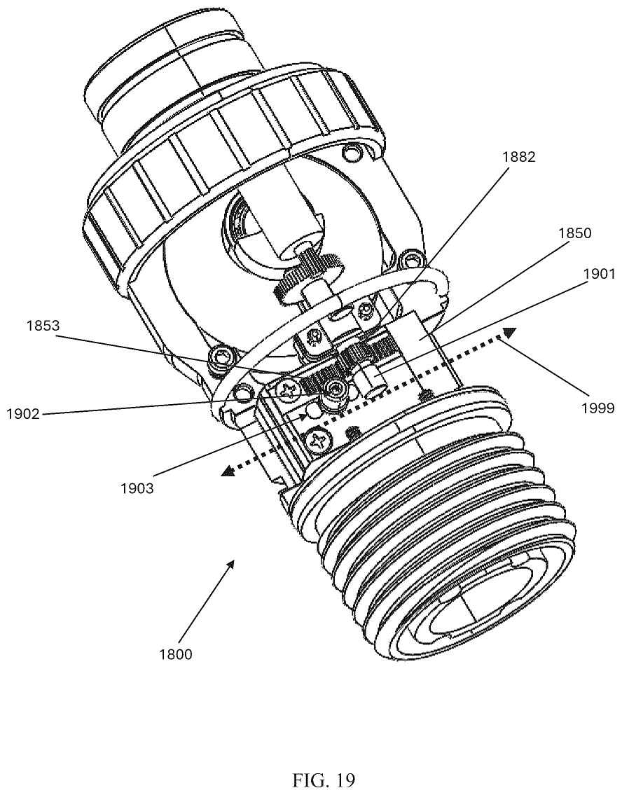

FIG. 19 is an illustration of a front perspective view of one embodiment of an interior of a direct drive adjustable stroke tattoo machine that may be adjusted manually or electrically. As shown in FIG. 19, adjustable limitation device 1602 may be removed and placed in one of several device holes 1903, which allows a user or manufacturer to adjust the range of the stroke length to be less than the full range. This is done by physically limiting the back and forth movement 1999 of rack platform 1850 via rack limiting nub 1601 and adjustable limitation device 1602. In addition, the movement 1999 of rack platform 1850 may be limited by sensor feedback, and physical constraints, such as the housing. The holes 1903 in other embodiments may be indentations or grooves.

In other embodiments, adjustable limitation device 1902 may be a spring loaded pressure device (such as a spring-loaded ball detent) that holds rack platform 1850 in place once the user, via a controller and via gear motor 1824, adjusts the stroke length. In this embodiment, rack limiting nub 1601 pushes adjustable limitation device 1902 from one hole 1903 to other holes, which are set at specific stroke length positions. In this embodiment holes 1903 are indentations (or detents) that matingly engage with a spring-loaded ball of adjustable limitation device 1902 such that rack platform 1850 is rigidly held at the desired position/stroke length.

As shown herein, the manual adjustment tattoo machines, such as tattoo machine 900, and the automatic/electrical adjustment tattoo machines, such as tattoo machine 100, may both also include rack limiting nubs and adjustable limitation devices, equivalents thereto, and the like.

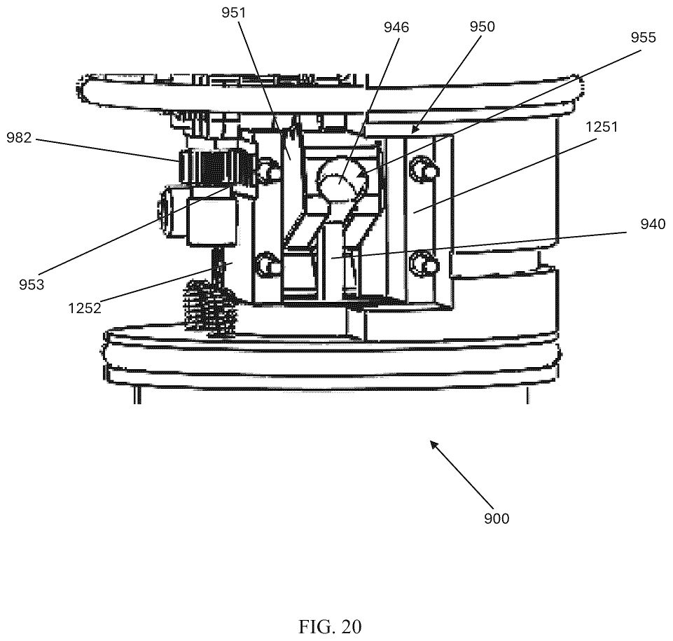

FIG. 20 is an illustration of a side perspective view of one embodiment of an interior of a direct drive adjustable stroke tattoo machine that may be adjusted manually or electrically. FIG. 20 shows a portion of the interior of tattoo machine 900, which may comprise second gear 982 and rack platform 950, which may comprise rack 953, guide arm 951, front support 1252 rear support 1251, and guide arm channel 955, plunger bar 940, which may comprise plunger bar guide arm end 946. FIG. 20 shows how the preferred embodiments, including tattoo machines 100, 900, 1800, allow for an easy and reliable adjustment of the stroke length. As second gear 982 turns left (as dictated by the user) rack 953 is moved to the right, which causes rack platform 950 and guide arm 951 to also move to the right. Since plunger bar 940 and plunger bar guide arm end 946 are not moveable from side to side (they are only moveable up and down as constrained by plunger channel 999), plunger bar slides within guide arm channel 955, such that plunger bar guide arm end 946 is closer to guide arm hinge connector 954. In this manner, the stroke length of plunger bar 940 is shortened, such that the needle or cartridges driven by plunger bar 940 do not go into a recipient's skin as deep. In this embodiment, if the user wants to lengthen the stroke length, the rack is moved to the left.

In various embodiments, the parts of the tattoo machines of the present disclosure are preferably made of composites, plastics, and metals. For example, the plunger bars may preferably be made of stainless steel. The cams, drive shafts, pins, connectors, and bearings may preferably be made of stainless steel or other strong metals. The connector rods may be made of aluminum. The guide arm may be acetal resin, such as polyoxymethylene (such as Delrin®). Other parts, housings, connectors, screws, grips, battery housings, supports, buttons, O-rings, and the like, may be made of rubber, composites, plastics, resins, metals, artificial, and natural materials, in order to conform to the durability and strength needed to accomplish the tattooing process.

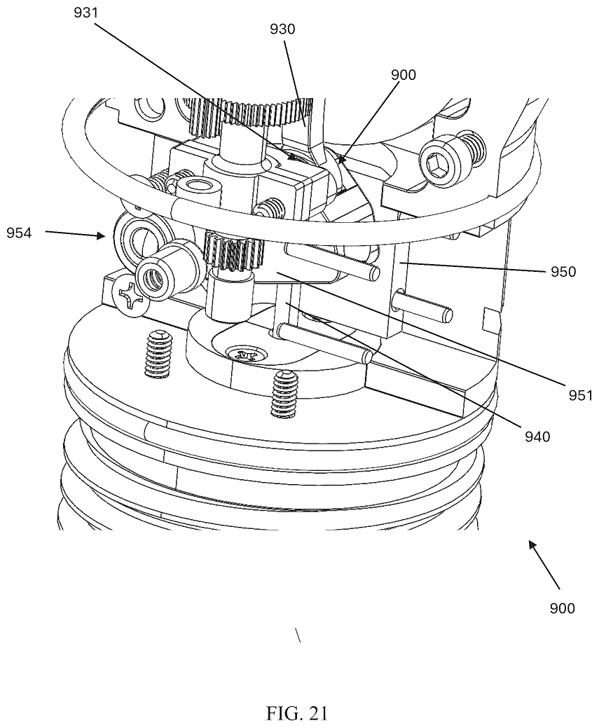

FIG. 21 is an illustration of a front and side perspective view of one embodiment of an interior of a direct drive adjustable stroke tattoo machine that may be adjusted manually or electrically. As shown in FIG. 21, tattoo machine 900 may have guide arm 951, which is secured to rack platform 950 via guide arm hinge connector 954. This allows guide arm 951 to be hingedly pushed in an up and down manner by connecting rod 930, which is connected to guide arm 850 via connecting rod pin connector 931. Guide arm 951, in turn, drives plunger bar 940 in a linear, up and down manner.

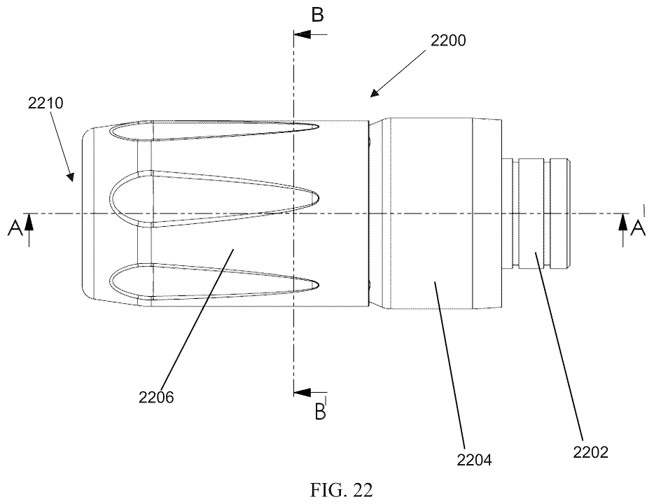

FIG. 22 is an illustration of a front view of another embodiment of a direct drive electronic adjustable stroke tattoo machine. As shown in FIG. 22, tattoo machine 2200 may comprise housing 2204, removable grip 2206, power connection end 2202 and needle end 221Q As shown, tattoo machine 2200 may be a compact pen style tattoo or microneedling machine.

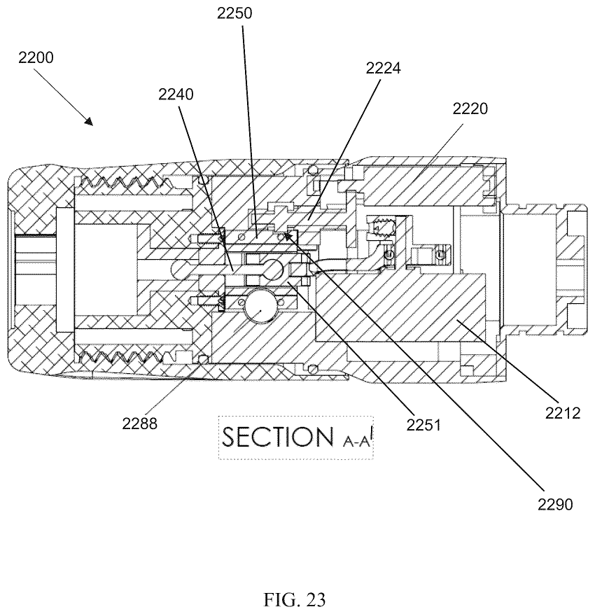

FIG. 23 is an illustration of a cross-section view of another embodiment of a direct drive electronic adjustable stroke tattoo machine. Tattoo machine 2200 is very similar to the tattoo machine shown in FIGS. 1-5 and works in essentially the same manner as shown and described previously. Tattoo machine 2200 may comprise drive motor 2212 gear drive motor 2220, gear drive 2224, rack platform 2250, which may comprise rack 2290, guide arm 2251, and magnet 2288, and plunger bar 2240. The embodiment shown in FIG. 23 has magnet 2288 embedded in rack platform 2250, such that as rack platform move in and out in a linear manner as driven by gear drive 2224, magnet 2288 is also moved in the same manner.

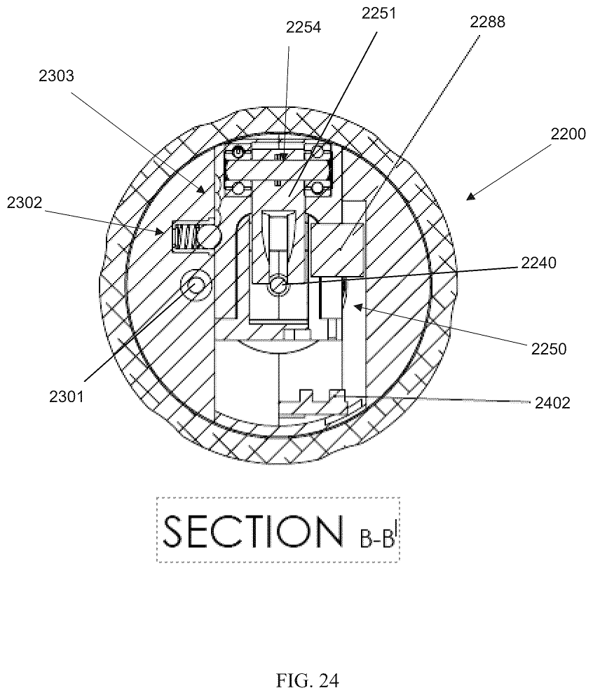

FIG. 24 is an illustration of another cross-section view of another embodiment of a direct drive electronic adjustable stroke tattoo machine. FIG. 24 shows one mechanism to precisely adjust the stroke length of tattoo machine 2200. As shown in FIG. 24, tattoo machine 2200 may comprise proximity sensor 2402 rack platform 2250, which may comprise guide arm 2251, guide arm hinge connector 2254, magnet 2288, pressure device 2302 and grooves 2303, plunger bar 2240, and pressure device movement nub 2301. As shown in FIG. 24, rack platform 2250 moves up and down in a linear manner. Pressure device 2302 and grooves 2303 work together as a ball detent system that holds rack platform in place once a stroke length is set by the user. As shown, pressure device 2302 may be a ball detent device or mechanism that allows pressure device 2302 to be moved by pressure device movement nub 2301 and then uses friction to hold rack platform 2250 in place as set by the user. Although only four grooves 2303 are shown, it should be understood that there may be no grooves or there may be dozens (30-60) of grooves or dimples to allow for numerous adjustment points to set and held without any accidental slippage.

Magnet 2288 moves linearly with rack platform 2250. As it moves in this manner, magnet 2280 gets closer to (or further away from) proximity sensor 2402. Proximity sensor 2402 monitors where magnet 2288 is in relation to proximity sensor 2402. This distance gets transmitted (wirelessly or by wire) to the battery/controller so the battery/controller always knows where rack platform 2250 is in relation to proximity sensor 2401. In this manner, battery/controller always knows the stroke length and how to adjust the stroke length to meet the requests/needs of the user or program. In the event that the battery/controller is swapped out or recharged the new or recharged battery controller can receive (and/or request) data related to the current stroke length from proximity sensor 2402.

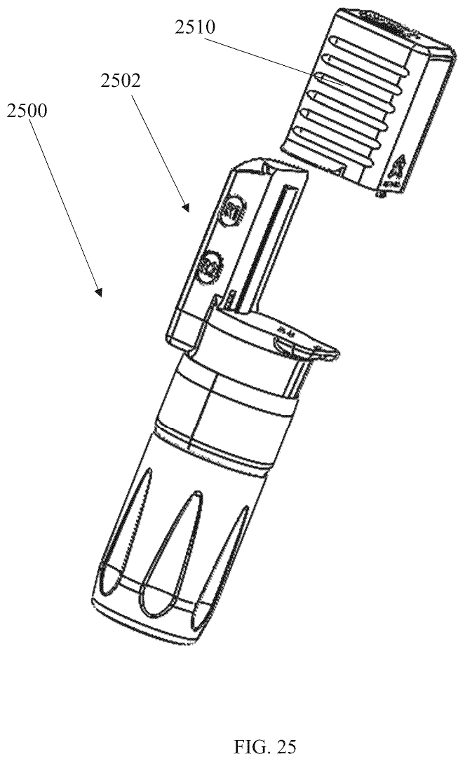

FIG. 25 is an illustration of a perspective view of another embodiment of a direct drive adjustable stroke tattoo machine. As shown in FIG. 25, tattoo machine 2500, which is substantially the same as tattoo machines 100, 900, and 1800, including having an adjustable stroke device, may comprise an integrated controller, which may include buttons, a display screen, and soft keys, that allow a user to adjust the voltage and the stroke length of the tattoo machine. FIG. 25 shows that the batter or power supply 2510 may be removeable and replaceable.

The manual stroke adjustment tattoo machines may also have sensors and pressure devices to assist the user in accurately informing the user of the stroke length and what adjustment points there may be.

Unless otherwise stated, all measurements, values, ratings, positions, magnitudes, sizes, locations, and other specifications, which are set forth in this specification, including in the claims that follow, are approximate, not exact. They are intended to have a reasonable range, which is consistent with the functions to which they relate and with what is customary in the art to which they pertain.

The foregoing description of the preferred embodiment has been presented for the purposes of illustration and description. While multiple embodiments are disclosed, still other embodiments will become apparent to those skilled in the art from the above detailed description, which shows and describes the illustrative embodiments. As will be realized, these embodiments are capable of modifications in various obvious aspects, all without departing from the spirit and scope of the present disclosure. Accordingly, the detailed description is to be regarded. As illustrative in nature and not restrictive. Also, although not explicitly recited, one or more additional embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing which has been stated or illustrated is intended or should be interpreted to cause a dedication of any component, step, feature, object, benefit, advantage, or equivalent to the public, regardless of whether it is or is not recited in the claims.

What is claimed is:

1. A tattoo machine, comprising:
a drive motor, which comprises a drive shaft;
a drive cam that is configured to matingly engage with said drive shaft;
a connecting rod that is configured to engage with said drive cam;
a stroke adjustment device, wherein said stroke adjustment device comprises a guide arm and a rack platform, which comprises a rack; and
a plunger bar, which is configured to engage with one or more tattoo needles, and which is configured to engage with said guide arm;
wherein said guide arm is configured to engage with said connecting rod and which is configured to engage said rack platform;
wherein said drive motor is configured to receive power from a power source;
wherein said stroke adjustment device is configured to adjust a stroke length of said tattoo machine.

2. The tattoo machine of claim 1, wherein said stroke adjustment device adjusts said stroke length electronically.

3. The tattoo machine of claim 1, wherein said stroke adjustment device adjusts said stroke length manually.

4. The tattoo machine of claim 1, wherein said stroke adjustment device adjusts said stroke length both electronically and manually.

5. The tattoo machine of claim 1, wherein said guide arm is hingedly connected to said rack platform, such that when said connecting rod is activated by said drive motor, said guide arm reciprocatingly moves said plunger bar in a linear manner.

6. The tattoo machine of claim 2, wherein said stroke adjustment device further comprises:
a drive gear motor, which is configured to receive power from said power source; and
a rack gear drive;
wherein said drive gear motor comprises a bottom gear;
wherein said rack gear drive comprises a first gear and a second gear;
wherein said bottom gear is configured to matingly engage with said first gear;
wherein said second gear is configured to matingly engage with said rack; and
wherein said drive gear motor is configured adjust said stroke length, such that when said drive gear motor rotates said bottom gear rotates, which in turn rotates said first gear, which in turn rotates said second gear, which in turn drives said rack, which moves said rack platform, which in turn repositions said guide arm, such that said stroke length is lengthened or shortened.

7. The tattoo machine of claim 6, wherein said drive gear motor is configured to accept one or more instructions to adjust said stroke length.

8. The tattoo machine of claim 7, wherein said power source is a battery; and
wherein said one or more instructions to adjust said stroke length are provided by said battery, which is operatively connected to said tattoo machine.

9. The tattoo machine of claim 3, wherein said stroke adjustment device further comprises:
a stroke adjustment ring;
a gear drive; and
a rack gear drive;
wherein said stroke adjustment ring comprises a plurality of stroke adjustment ring gear teeth;
wherein said gear drive comprises a top gear and a bottom gear;
wherein said rack gear drive comprises a first gear and a second gear;
wherein said stroke adjustment ring gear teeth are configured to matingly engage with said top gear;
wherein said bottom gear is configured to matingly engage with said first gear;
wherein said second gear is configured to matingly engage with said rack; and
wherein said stroke adjustment ring is configured to be turned to adjust said stroke length, such that when said stroke adjustment ring is rotated, and thus said stroke adjustment ring gear teeth are rotated, said stroke adjustment ring gear teeth rotates said top gear, which in turn rotates said bottom gear, which in turn rotates said first gear, which in turn rotates said second gear, which in turn drives said rack, which moves said rack platform, which in turn repositions said guide arm, such that said stroke length is lengthened or shortened.

10. The tattoo machine of claim 4, wherein said stroke adjustment device further comprises:
a stroke adjustment ring;
a gear drive/drive gear motor; and
a rack gear drive;
wherein said stroke adjustment ring comprises a plurality of stroke adjustment ring gear teeth;
wherein said a gear drive/drive gear motor comprises a top gear and a bottom gear;
wherein said rack gear drive comprises a first gear and a second gear;
wherein said stroke adjustment ring gear teeth are configured to matingly engage with said top gear;
wherein said bottom gear is configured to matingly engage with said first gear;
wherein said second gear is configured to matingly engage with said rack; and
wherein said stroke adjustment ring is configured to be turned to manually adjust said stroke length, such that said stroke adjustment ring, and thus said stroke adjustment ring gear teeth, rotates, which in turn rotates said top gear, which in turn rotates said bottom gear, which in turn rotates said first gear, which in turn rotates said second gear, which in turn drives said rack, which moves said rack platform, which in turn repositions said guide arm, such that said stroke length is lengthened or shortened; and
wherein said gear drive/drive motor gear is configured to electronically adjust said stroke length, such that said gear drive/drive motor gear rotates said bottom gear, which in turn rotates said first gear, which in turn rotates said second gear, which in turn drives said rack, which moves said rack platform, which in turn repositions said guide arm, such that said stroke length is lengthened or shortened.

11. The tattoo machine of claim 10, wherein said gear drive/drive motor gear is configured to accept one or more instructions to electronically adjust said stroke length.

12. The tattoo machine of claim 7, wherein said power source is a battery; and
wherein said one or more instructions to adjust said stroke length are provided by said battery, which is operatively connected to said tattoo machine.

13. An electronic stroke adjustment tattoo machine, comprising:
a drive motor, which comprises a drive shaft;
a drive cam that is configured to matingly engage with said drive shaft;
a connecting rod that is configured to engage with said drive cam;
a stroke adjustment device, wherein said stroke adjustment device comprises a guide arm, a drive gear motor; a rack gear drive; and a rack platform;
wherein said guide arm is configured to engage with said connecting rod and which is configured to engage said rack platform;
a plunger bar, which is configured to engage with one or more tattoo needles, and which is configured to engage with said guide arm;
wherein said drive motor is configured to receive power from a power source;
wherein said guide arm is hingedly connected to said rack platform, such that when said connecting rod is activated by said drive motor, said guide arm reciprocatingly moves said plunger bar in a linear manner;
wherein said stroke adjustment device is configured to adjust a stroke length of said tattoo machine.

14. The electronic stroke adjustment tattoo machine of claim 13, wherein said rack platform comprises a rack;
wherein said drive gear motor comprises a bottom gear;
wherein said rack gear drive comprises a first gear and a second gear;
wherein said bottom gear is configured to matingly engage with said first gear; and
wherein said second gear is configured to matingly engage with said rack.

15. The electronic stroke adjustment tattoo machine of claim 14, wherein said drive gear motor is configured to adjust said stroke length, such that said drive gear motor rotates said bottom gear, which in turn rotates said first gear, which in turn rotates said second gear, which in turn drives said rack, which moves said rack platform, which in turn repositions said guide arm, such that said stroke length is lengthened or shortened.

16. The electronic stroke adjustment tattoo machine of claim 15, wherein said drive gear motor is configured to accept one or more instructions to adjust said stroke length.

17. The electronic stroke adjustment tattoo machine of claim 16, wherein said power source is a battery; and
wherein said one or more instructions to adjust said stroke length are provided by said battery, which is operatively connected to said tattoo machine.

18. A manual stroke adjustment tattoo machine, comprising:
a drive motor, which comprises a drive shaft;
a drive cam that is configured to matingly engage with said drive shaft;
a connecting rod that is configured to engage with said drive cam;
a stroke adjustment device, wherein said stroke adjustment device comprises a stroke adjustment ring, a gear drive, a rack gear drive; a guide arm, and a rack platform;
wherein said guide arm is configured to engage with said connecting rod and which is configured to engage said rack platform;
a plunger bar, which is configured to engage with one or more tattoo needles, and which is configured to engage with said guide arm;
wherein said drive motor is configured to receive power from a power source;
wherein said guide arm is hingedly connected to said rack platform, such that when said connecting rod is activated by said drive motor, said guide arm reciprocatingly moves said plunger bar in a linear manner; and
wherein said stroke adjustment device is configured to adjust a stroke length of said tattoo machine.

19. The manual stroke adjustment tattoo machine of claim 18, wherein said rack platform comprises a rack;
wherein said stroke adjustment ring comprises a plurality of stroke adjustment ring gear teeth;
wherein said gear drive comprises a top gear and a bottom gear;
wherein said rack gear drive comprises a first gear and a second gear;
wherein said stroke adjustment ring gear teeth are configured to matingly engage with said top gear;
wherein said bottom gear is configured to matingly engage with said first gear; and
wherein said second gear is configured to matingly engage with said rack.

20. The manual stroke adjustment tattoo machine of claim 19, wherein said stroke adjustment ring is configured to be turned to adjust said stroke length, such that said stroke adjustment ring, and thus said stroke adjustment ring gear teeth, rotates, which in turn rotates said top gear, which in turn rotates said bottom gear, which in turn rotates said first gear, which in turn rotates said second gear, which in turn drives said rack, which moves said rack platform, which in turn repositions said guide arm, such that said stroke length is lengthened or shortened; and
wherein an upper end of said stroke length may be adjusted or limited.
